(19) United States
(12) Patent Application Publication
(10) Pub. No.: US 2023/0100211 A1
(43) Pub. Date: Mar. 30, 2023

(54) PROCESS FOR THE PREPARATION OF SODIUM 4-(2-((1E,3E,5E,7Z)-7-(1,1-DIMETHYL-3-(4-SULFONATOBUTYL)-1H-BENZO[e]INDOL-2(3H)-YLIDENE) HEPTA-1,3,5-TRIENYL)-1,1-DIMETHYL-1H-BENZO[e]INDOLIUM-3-YL) BUTANE-1-SULFONATE (INDOCYANINE GREEN)

(71) Applicant: BIOPHORE INDIA PHARMACEUTICALS PVT. LTD., HYDERABAD (IN)

(72) Inventors: MANIK REDDY PULLAGURLA, HYDERABAD (IN); JAGADEESH BABU RANGISETTY, HYDERABAD (IN)

(21) Appl. No.: 17/972,008

(22) Filed: Oct. 24, 2022

Related U.S. Application Data
(62) Division of application No. 16/398,525, filed on Apr. 30, 2019.

(30) Foreign Application Priority Data
May 2, 2018 (IN) 201841016487

(51) Int. Cl. C07D 209/60; C09B 67/48; C09B 23/08

(52) U.S. Cl. CPC C07D 209/60; C09B 67/0025; C09B 23/086; C07B 2200/13

(57) ABSTRACT

A process for the preparation of substantially pure Indocyanine green of formula with purity greater than 99.0% is provided. More particularly, the embodiments relate to the process for the preparation of Indocyanine green of formula and its intermediates thereof. It further provides crystalline form I of Indocyanine green of formula and process thereof.

### CROSS-REFERENCE TO RELATED APPLICATIONS

This application is a divisional of U.S. patent application Ser. No. 16/398,525, having a filing date of Apr. 30, 2019, which claims priority to Indian Application No. 201841016487, having a filing date of May 2, 2018, the entire contents both of which are hereby incorporated by reference.

### FIELD OF TECHNOLOGY

The following relates to a process for the preparation Indocyanine green (ICG) of formula (1). More particularly, the following relates to a process for the preparation of Indocyanine green (ICG) of formula (1) in a substantially pure form with a purity greater than 99.0%. The following also relates to crystalline form I of Indocyanine green (ICG) of formula (1) and a process for the preparation thereof.

### BACKGROUND

Indocyanine green (ICG) of formula (1) is a cyanine dye used in medical diagnostics. It is used for determining cardiac output, hepatic function, and liver blood flow, and for ophthalmic angiography. Chemically, it is sodium 4-[2-[(1E,3E,5E,7Z)-7-[1,1-dimethyl-3-(4-sulfonatobutyl)benzo[e]indol-2-ylidene]hepta-1,3,5-trienyl]-1,1-dimethylbenzo[e]indol-3-ium-3-yl]butane-1-sulfonate.

Several synthetic routes were reported in patents for the preparation of Indocyanine green of formula (1). The contents of which are hereby incorporated as reference in their entirety.

U.S. Pat. No. 5,750,722 describes the synthesis of Indocyanine green of formula (1) by reacting 1,1,2-trimethyl-1H-benzo[e]indole of formula (5) with 1,4-butane sultone to form intermediate of formula (4), which on treatment with N-((2E,4E)-5-(phenylamino) penta-2,4-dienylidene) aniline hydrochloride of formula (3) in presence of triethylamine and sodium acetate obtained Indocyanine green of formula (1).

U.S. Pat. No. 2,895,955 discloses the preparation of iodide disodium salt derivative of Indocyanine green of formula (1) by treating 1,1,2-trimethyl-1H-benzo[e]indole with 1,4-butane sultone to form intermediate of formula (6) 4-(1,1,2-trimethyl-1H-benzo[e]indolium-3-yl) butane-1-sulfonate. Further reaction of intermediate of formula (6) with N-((2E,4E)-5-(phenyl amino) penta-2,4-dienylidene) aniline hydrochloride in presence of sodium iodide forms iodide disodium salt derivative of Indocyanine green of formula (1).

Most of the reported methods describe the synthesis of intermediates and different derivatives of Indocyanine green, but the preparation of pure Indocyanine green is not reported. Moreover, the purification process and purity of intermediates are not reported in any of the prior art methods though they have discussed about the yields.

The embodiments therefore aim at providing a process for preparation of Indocyanine green of formula (1) and its intermediates thereof with purity more than 99.0% using simple purification methods.

### SUMMARY

An aspect relates to a process for the preparation of substantially pure Indocyanine green of formula (1), having purity greater than 99.0% by High-performance liquid chromatography (HPLC), greater than 99.5% and total impurities less than 1.0% or less than 0.5%.

Another aspect of embodiments of the invention is to provide a process for preparing crystalline form I of Indocyanine green of formula (1) with moisture content less than 2%.

Another aspect of embodiments of the invention is to provide purification process to remove the process related impurities which include impurity A, impurity B, impurity C and impurity D.

Another aspect of embodiments of the invention is to provide process for the purification of Indocyanine green of formula (1) to obtain substantially pure Indocyanine green of formula (1) having purity greater than 99.0%, or greater than 99.5%, which comprises:
1) suspending Indocyanine green of formula (1) in a suitable solvents or mixture thereof;
2) heating the reaction mixture at suitable temperature;
3) filtering the reaction mixture at suitable temperature; and
4) isolating from a suitable solvents or mixture thereof.

Indocyanine green of formula (1) obtained in the above procedure is having purity greater than 99.0% (by HPLC), or greater than 99.5% and total impurities less than 1.0%, or less than 0.5%.

Another aspect of embodiments of the invention is to provide Indocyanine green of formula (1) with elemental impurity having lead level less than 0.5 ppm and Arsenic level less than 1.5 ppm, combination of lead and arsenic is less than 2 ppm.

Accordingly, it is an aspect of embodiments of the present invention to provide a process for the preparation of Indocyanine green of formula (1), which comprises of:
a) reacting 1,1,2-trimethyl-1H-benzo[e]indole of formula (5)

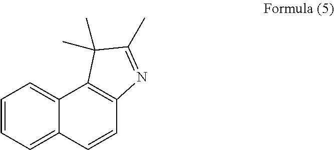 [SMILES: CC1=Nc2ccc3ccccc3c2C1(C)C]

with 1,4-butane sultone to form 4-(1,1,2-trimethyl-1H-benzo[e]indolium-3-yl) butane-1-sulfonate of formula (4);

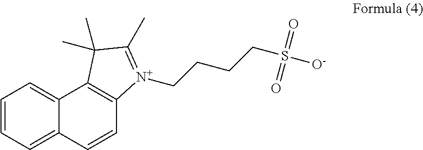 [SMILES: CC1=[N+](CCCCS(=O)(=O)[O-])c2ccc3ccccc3c2C1(C)C]

b) treating formula (4) with E-N-((2E,4E)-5-(phenyl amino) penta-2,4-dienylidene) aniline hydrochloride of formula (3)

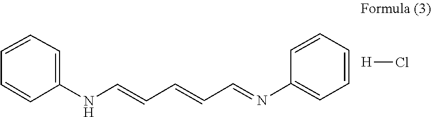

in presence of acetic anhydride to form 4-(1,1-dimethyl-2-((1E,3E,5E)-6-(N-phenyl acetamido) hexa-1,3,5-trienyl)-1H-benzo[e]indolium-3-yl) butane-1-sulfonate of formula (2);

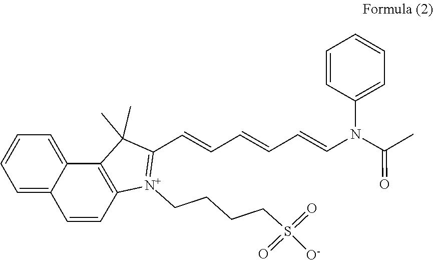 [SMILES: CC(=O)N(/C=C/C=C/C=C/C1=[N+](CCCCS(=O)(=O)[O-])c2ccc3ccccc3c2C1(C)C)c1ccccc1]

c) reacting formula (2) with formula (4) in presence of a base to obtain 4-amino-N-(2-(diethylamino)ethyl)benzamide hydrochloride of formula (1); and
d) purifying 4-amino-N-(2-(diethylamino)ethyl)benzamide hydrochloride of formula (1).

Another aspect of embodiments of the invention is to provide process for the preparation of N-((2E,4E)-5-(phenylamino) penta-2,4-dienylidene) aniline hydrochloride of formula (3) as depicted in scheme-2, which comprises:
i. reacting 1-chloro-2,4-dinitrobenzene of formula (7)

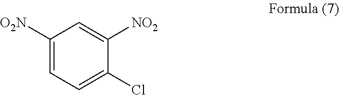 [SMILES: O=[N+]([O-])c1ccc(Cl)c([N+](=O)[O-])c1]

with pyridine to form 1-(2,4-dinitrophenyl)pyridinium chloride of formula (6);

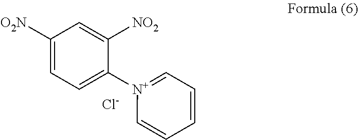 [SMILES: O=[N+]([O-])c1ccc(-[n+]2ccccc2)c([N+](=O)[O-])c1 ; [Cl-]]

ii. reacting formula (6) with methanolic aniline to form N-((2E,4E)-5-(phenyl amino) penta-2,4-dienylidene) aniline hydrochloride of formula (3); and

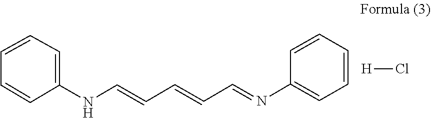

iii. purifying N-((2E,4E)-5-(phenyl amino) penta-2,4-dienylidene) aniline hydrochloride of formula (3).

In another aspect of embodiments of the invention, the formula (3) produced in embodiments of the present invention is having purity greater than 90.0% by HPLC.

### BRIEF DESCRIPTION OF DRAWINGS

Some of the embodiments will be described in detail, with references to the following Figures, wherein like designations denote like members, wherein:

FIG. 1 is characteristic X-ray powder diffraction pattern (XRD) of crystalline form I of Indocyanine green of formula (1); and

FIG. 2 is characteristic Infrared spectrum of Indocyanine green of formula (1).

### DETAILED DESCRIPTION OF THE INVENTION

Accordingly, embodiments of the present invention provide a process for the preparation of substantially pure Indocyanine green of formula (1) with purity greater than 99.0%, or greater than 99.5% purity by HPLC.

In one aspect, embodiments of the present invention provide a process for preparation of Indocyanine green of formula (1) as depicted in following scheme-1.

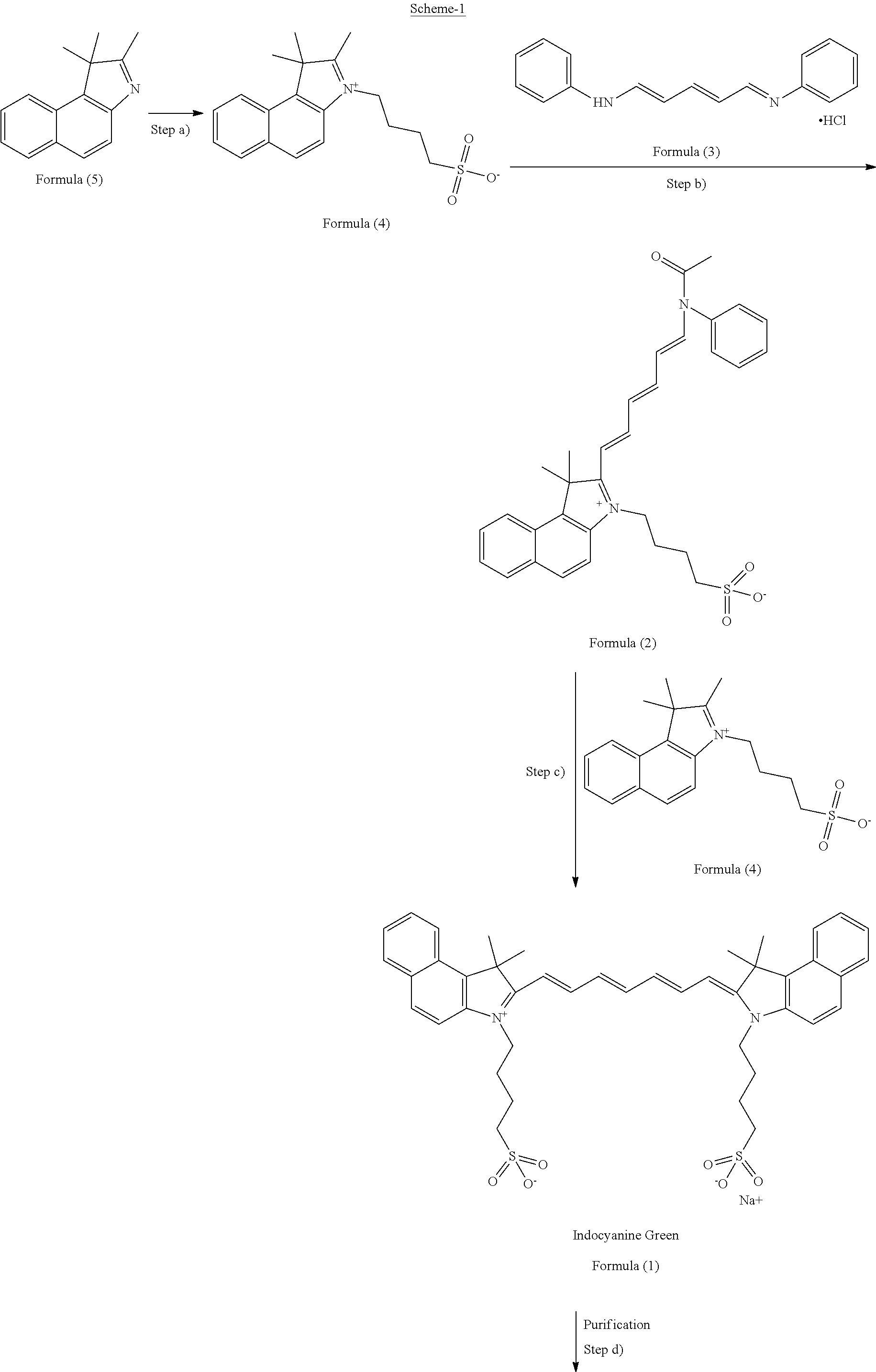

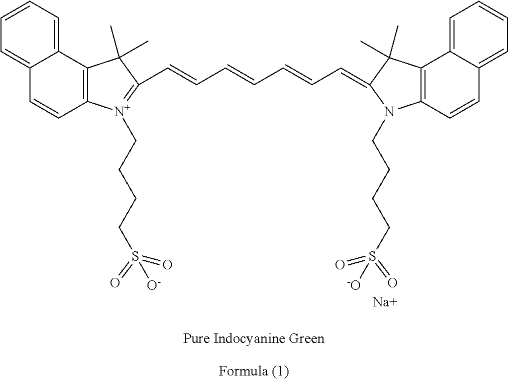

Step a) proceeds with reacting 1,1,2-trimethyl-1H-benzo[e]indole of formula (5) with 1,4-butane sultone in a suitable solvent at a suitable temperature to obtain 4-(1,1,2-trimethyl-1H-benzo[e]indolium-3-yl) butane-1-sulfonate of formula (4).

The said reaction can be carried out at a suitable temperature of 120-160° C., or at 140-150° C. The reaction mixture can be then cooled to 0-40° C., or to 25-30° C. The suitable solvent used can be selected from an aprotic solvent.

The intermediate 4-(1,1,2-trimethyl-1H-benzo[e]indolium-3-yl) butane-1-sulfonate of formula (4) can be purified by suspending formula (4) in a mixture of suitable protic or aprotic solvents and heating to a suitable temperature. The reaction mixture can be cooled to suitable temperature of 0-15° C. The obtained solid mass can be washed with an aprotic solvent to obtain purity greater than 90%, which is not disclosed in any previous reports. The suitable temperature can be ranges from 50-90° C., or to 65-85° C.

The protic solvents used in the purification of formula (4) were selected from a group comprising of methanol, ethanol, isopropyl alcohol (IPA), n-propanol, n-butanol, water or the like, isopropyl alcohol, methanol or mixtures thereof were used in embodiments of the present invention.

The aprotic solvents used in step a) and in the purification of formula (4) were selected from a group comprising of hexane, cyclohexane, toulene, xylene, tetrahydrofuran, acetone, acetonitrile, 1,4-dioxane, diethyl ether, dichloromethane, ethyl acetate, N, N-dimethylformamide, methyl tertiary butyl ether or the like, xylene and acetone were used.

Step b) proceeds with the condensation of formula (4) with of formula (3) in presence of acetic anhydride. The reaction can be carried out at temperature of 100-170° C., to 130-140° C. to form crude 4-(1,1-dimethyl-2-((1E,3E,5E)-6-(N-phenyl acetamido) hexa-1,3,5-trienyl)-1H-benzo[e]indolium-3-yl) butane-1-sulfonate of formula (2).

The reaction can be carried out by adding equal volumes of acetic anhydride and acetic acid to formula (2) at 25-30° C. Heating the reaction mixture to 100-150° C., or to 120-130° C., then cooling to 0-30° C., or to 10-15° C. provides of formula (2), which is further purified from a suitable aprotic solvent to obtain purity greater than 90%.

The suitable aprotic solvents used in step b) and in the purification of formula (2) can be selected from a group comprising of hexane, cyclohexane, toulene, xylene, tetrahydrofuran, acetone, acetonitrile, 1,4-dioxane, diethyl ether, dichloromethane, ethyl acetate, N, N-dimethylformamide, methyl tertiary butyl ether or the like, methyl tertiary butyl ether and acetone were used in embodiments of the present invention.

Step c) involves the reacting formula (4) with formula (2) in a suitable protic solvent in presence of a base. Step c) can be performed at a temperature ranging from 50-70° C., or at 60-65° C. after completion of the reaction, the reaction mass can be distilled off and a suitable protic or aprotic solvent can be used for the isolation of Indocyanine green of formula (1).

The protic solvents used in step c) may be selected from a group comprising of methanol, ethanol, isopropyl alcohol (IPA), n-propanol, n-butanol, water or the like, isopropyl alcohol, methanol and or mixtures thereof were used in embodiments of the present invention. The aprotic solvent used in step c) was acetone.

The base used in step c) may be selected from a group comprising of triethylamine, pyridine, ammonia, potassium carbonate, sodium carbonate, potassium bicarbonate, sodium bicarbonate, potassium hydroxide or sodium hydroxide or the like. Triethylamine was used in embodiments of the present invention.

Step d) involves purification of Indocyanine green of formula (1).

In another aspect, embodiments of the present invention provide process for the purification of Indocyanine green (1), which comprises of:
1) suspending Indocyanine green of formula (1) in a suitable solvents or mixture thereof;
2) heating the reaction mixture at suitable temperature;
3) filtering the reaction mixture at suitable temperature; and
4) isolating from a suitable solvents or mixture thereof.

In another aspect, the purification of Indocyanine green (1) can be carried out by suspending the crude in a mixture of suitable solvents. The reaction mixture can be heated to a temperature ranges from about 50-90° C., or to 60-80° C. Purification can be carried out using a protic solvent or mixtures thereof. The present method is advantageous over other reported methods as it reduces the by-product formation and cost of raw materials used.

In another aspect, the suitable solvents used in the purification of Indocyanine green of formula (1) were selected from a group comprising of acetone, methanol, ethanol, isopropyl alcohol (IPA), n-propanol, n-butanol, water or the like, isopropyl alcohol, methanol and or mixtures thereof.

In another aspect, embodiments of the present invention provide process for the synthesis of intermediate N-((2E,4E)-5-(phenyl amino) penta-2,4-dienylidene) aniline hydrochloride of formula (3) with purity greater than 90.0% by HPLC as shown in scheme-2, which comprises of the following steps:

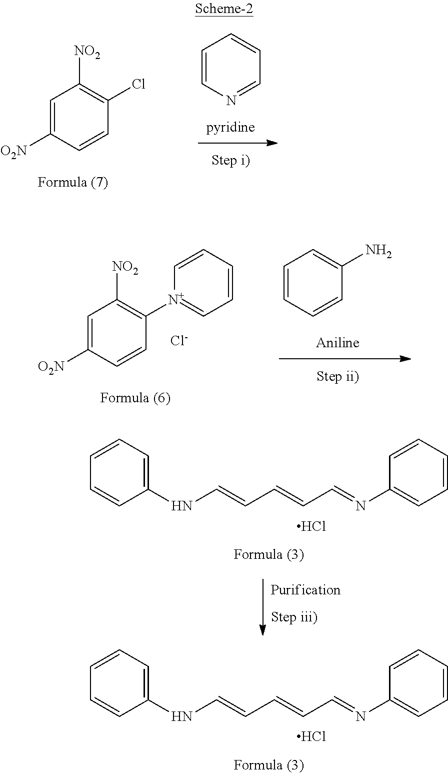

Step i) proceeds with addition of 1-chloro-2,4-dinitrobenzene of formula (7) to pyridine, dissolved in a suitable aprotic solvent. The resulting solution was then refluxed at 50-70° C., or at 60-65° C., then cooled to 0-30° C., or to 25-30° C. The resulting solid so obtained was washed with an aprotic solvent and dried under vacuum to obtain 1-(2,4-dinitrophenyl) pyridinium chloride of formula (6)

Step ii) involves reacting formula (6) with aniline in presence of a suitable protic solvent to obtain (E)-N-((2E,4E)-5-(phenyl amino) penta-2,4-dienylidene) aniline hydrochloride of formula (3)

Step iii) proceeds with the purification of formula (3) by dissolving in a mixture of aprotic and protic solvents to obtain pure (E)-N-((2E,4E)-5-(phenyl amino) penta-2,4-dienylidene) aniline hydrochloride of formula (3) with purity greater than 90%. The reaction mixture was heated to a temperature ranges from 50-70° C., or to 60-65° C. then cooling to 0-30° C., or to 25-30° C.

The suitable aprotic solvents used in process of scheme-2 were selected from a group comprising of xylene, benzene, toluene, acetone, methyl tertiary butyl ether, dichloromethane, or the like. Acetone was used. The suitable protic solvents used in scheme-2 were selected from a group comprising of methanol, ethanol, isopropyl alcohol or the like, methanol and isopropyl alcohol were used in embodiments of the present invention.

The following facilitates the easy removal of many undesired impurities and maintains the pH of the product between 5.0 to 7.0 by providing high purity Indocyanine green of formula (1)

Indocyanine green of formula (1) obtained by the purification process of embodiments of the present invention is substantially pure and has purity greater than 99.0%, greater than 99.5% and total impurities less than 1.0% and less than 0.5%.

In another aspect of embodiments of the invention, Indocyanine green of formula (1) obtained by the purification of embodiments of the present invention is having total impurities less than 1.0% and or less than 0.5% and each of impurity A, B, C and D is less than 0.15% (w/w).

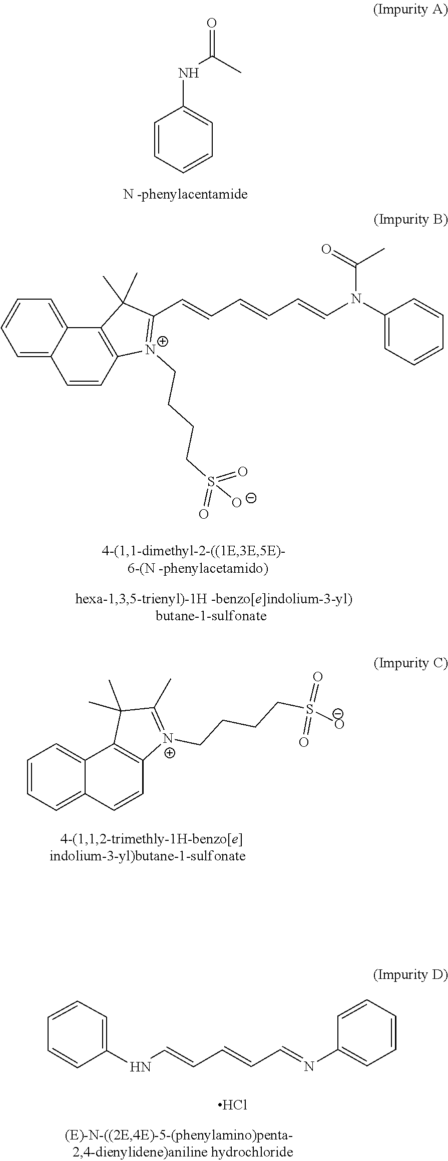

Indocyanine green of formula (1) obtained by the purification process of embodiments of the present invention is substantially pure and has purity greater than 99.0%, or greater than 99.5% measured by HPLC and which comprise lead less than 0.5 ppm and Arsenic less than 1.5 ppm, which forms another aspect of embodiments of the invention.

Indocyanine green of formula (1) obtained by the purification process of embodiments of the present invention is comprising total impurities less than 1.0% and or less than 0.5%, measured by HPLC and combination of lead and Arsenic metals less than 2 ppm.

In another aspect embodiments of the present invention provides Indocyanine green of formula (1) having acetic acid content less than 5.0% or less than 4.0% (w/w) by HPLC.

In addition, Indocyanine green of formula (1) synthesized according to embodiments of the present invention is having metal impurities as shown in table-2 and forms yet another object of embodiments of the invention.

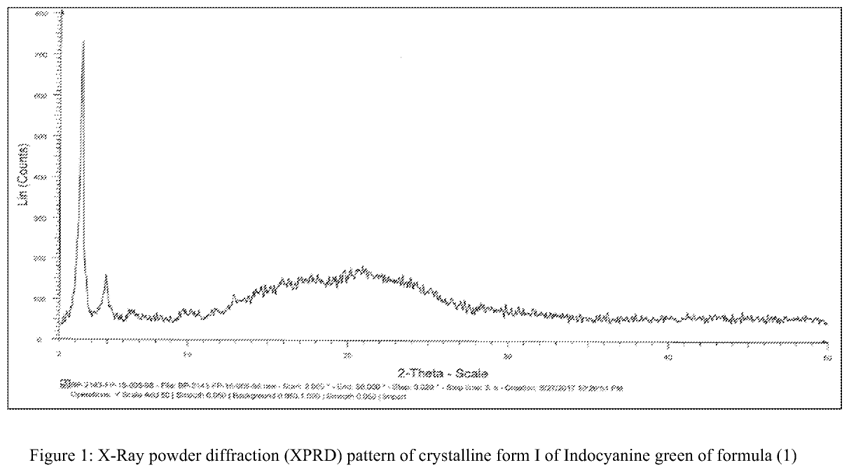

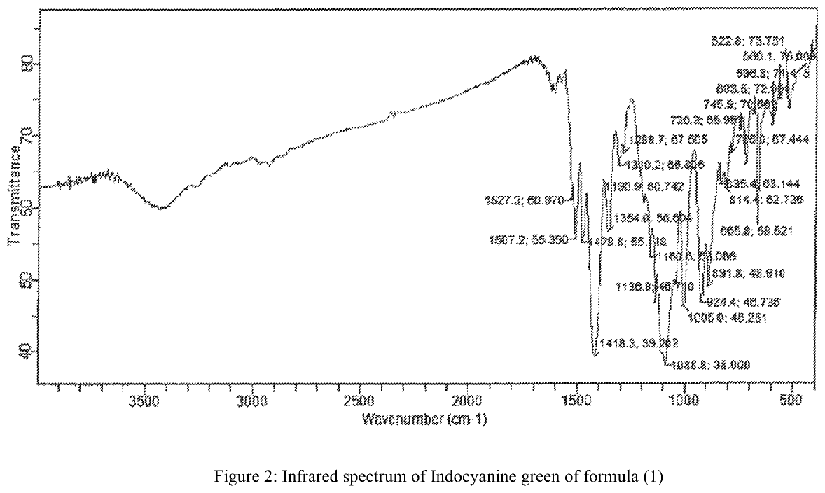

The crystalline form I of Indocyanine green of formula (1) obtained after purification is characterized by the X-ray powder diffraction (XRPD) pattern as shown in FIG. 1 and table-1 and infrared (IR) spectrum as shown in FIG. 2.

TABLE-1
X-ray diffraction data of Indocyanine green of formula (1)

| S.No | 2 theta degree. | Relative Intensity % (I/I₀) |
|---|---|---|
| 1 | 3.30 | 100 |
| 2 | 4.86 | 23.7 |

TABLE-2

| S. | | Elemental results of | ICH safety limit |
|---|---|---|---|
| No | Element | Indocyanine green (ppm) | (ppm) |
| 1. | Cadmium | 0.001 | 0.2 |
| 2. | Mercury | Not detected | 0.3 |
| 3. | Cobalt | 0.039 | 0.5 |
| 4. | Vanadium | 0.06 | 1 |
| 5. | Nickel | 2.2 | 5 |
| 6. | Thallium | Not detected | 0.8 |
| 7. | Gold | Not detected | 10 |
| 8. | Palladium | Not detected | 1 |
| 9. | Iridium | Not detected | 1 |
| 10. | Osmium | 0.001 | 1 |
| 11. | Rhodium | Not detected | 1 |
| 12. | Ruthenium | Not detected | 1 |
| 13. | Selenium | Not detected | 8 |
| 14. | Silver | Not detected | 1 |
| 15. | Platinum | Not detected | 1 |
| 16. | Lithium | Not detected | 25 |
| 17. | Antimony | 0.001 | 9 |
| 18. | Barium | Not detected | 70 |
| 19. | Molybdenum | 0.355 | 150 |
| 20. | Copper | 0.10 | 30 |
| 21. | Tin | Not detected | 60 |
| 22. | Chromium | 10 | 110 |

The following examples further illustrate embodiments of the present invention but should not be construed in any way as to limit its scope.

### Examples

### Example 1: Preparation of 4-(1,1,2-trimethyl-1H-benzo[e]indolium-3-yl) butane-1-sulfonate (4)

100 g of 1,1,2-trimethyl-1H-benzo[e]indole of formula (5) was taken in 600 mL of xylene. 88.6 mL of 1,4-butane sultone was added and stirred for 5-10 min. The reaction mass was heated for 12 hrs at 140-150° C. To this 88.6 mL of 1,4-butane sultone was added and stirred for another 6 hrs at 140-150° C. On completion of reaction, the reaction mixture was cooled to 60-65° C. and acetone was added. The reaction mixture was maintained at 60-65° C. for 30-45 min and cooled to 25-30° C. The precipitated solid was filtered and washed with acetone to yield 4-(1,1,2-trimethyl-1H-benzo[e]indolium-3-yl) butane-1-sulfonate of formula (4). Yield: 150 g.

Purification of 4-(1,1,2-trimethyl-1H-benzo[e]indolium-3-yl) butane-1-sulfonate (4) 150 g of formula (4) was taken in a mixture of 1500 mL isopropyl alcohol and 150 mL methanol and stirred for 5-10 mins at 25-30° C. The reaction mixture was heated for 30-45 mins at 65-85° C. then cooled to 10-15° C. The reaction mixture was filtered and washed with acetone. The obtained solid was dried under vacuum to obtain pure formula (4). Yield: 72%; Purity by HPLC: 99.8%

### Example 2: Preparation of 4-(1,1-dimethyl-2-((1E,3E,5E)-6-(N-phenyl acetamido) hexa-1,3,5-tri enyl)-1H-benzo[e]indolium-3-yl) butane-1-sulfonate (2)

100 g of formula (4) was taken in 615 mL of acetic anhydride. To this (E)-N-((2E,4E)-5-(phenylamino) penta-2,4-dienylidene) aniline hydrochloride of formula (3) and 77 mL of acetic anhydride were added at 25-30° C. The resulting solution was heated at 130-140° C. for 1.5 hour, then cooled to 10-15° C. for 30-45 min. The obtained solid was filtered and washed with 200 mL of methyl tertiary butyl ether then dried under vacuum to obtain 4-(1,1-dimethyl-2-((1E,3E,5E) (N-phenyl acetamido) hexa-1,3,5-trienyl)-1H-benzo[e]indolium-3-yl) butane-1-sulfonate of formula (2).Yield: 104 g.

Purification of 4-(1,1-dimethyl-2-((1E,3E,5E)-6-(N-phenyl acetamido) hexa-1,3,5-trienyl)-1H-benzo[e]indolium-3-yl) butane-1-sulfonate (2).

390 mL of acetic anhydride and 390 mL of acetic acid were added to 104 g of formula (2) at 25-30° C. The resulting solution was heated at 120-130° C. for 30-45 min, then cooled to 10-15° C. The solid was filtered and washed with acetone. The resulting solid was dried under vacuum to obtain pure 4-(1,1-dimethyl-2-((1E,3E,5E)-6-(N-phenyl acetamido) hexa-1,3,5-trienyl)-1H-benzo[e]indolium-3-yl) butane-1-sulfonate (2).

### Yield: 72%; Purity by HPLC: 99.9%.

### Example 3: Preparation of 1-(2,4-dinitrophenyl)pyridinium chloride (6)

100 g of 2,4-dinitro chlorobenzene of formula (7) was dissolved in 200 mL of acetone and stirred for 10-15 min. 33.65 g of pyridine was dissolved in 50 mL of acetone was slowly added to the above reaction mixture and heated for 12-14 hrs at 60-65° C. The resulting solution was then cooled to 25-30° C., filtered and the solid so formed was washed with 200 mL of acetone. The resulting solid was dried under vacuum to obtain 1-(2,4-dinitrophenyl)pyridinium chloride of formula (6). Yield: 83%.

### Example 4: Preparation of (E)-N-((2E,4E)-5-(phenyl amino) penta-2,4-dienylidene) aniline hydrochloride (3)

100 g of formula (6) was dissolved in 220 mL of 80% aqueous methanol and stirred for 10-15 min. A solution of 40 g of aniline dissolved in 300 mL of 80% aqueous methanol was then added to the above solution at 25-30° C. and stirred for 2 hrs. The reaction mass was then cooled to 10-15° C., stirred for 30-45 minutes and filtered. The solid so obtained was collected and the filtrate was stirred for 12-15 hrs at 25-30° C. The total solid was combined and dried under vacuum below 35° C. to obtain (E)-N-((2E,4E)-5-(phenyl amino) penta-2,4-dienylidene) aniline hydrochloride of formula (3). Yield: 80 g.

Purification of (E)-N-((2E,4E)-5-(phenylamino) penta-2,4-dienylidene) aniline hydrochloride (3).

80 g of N-((2E,4E)-5-(phenyl amino) penta-2,4-dienylidene) aniline hydrochloride of formula (3) was taken in a mixture of 800 mL of methyl tertiary-butyl ether and 40 mL of isopropyl alcohol and heated for 1 hr at 60-65° C. The resulting solution was cooled to 25-30° C. and stirred for 2 hrs. The solid so precipitated was filtered and washed with methyl tertiary butyl ether and dried under vacuum below 35° C. to obtain pure (E)-N-((2E,4E)-5-(phenyl amino) penta-2,4-dienylidene) aniline hydrochloride of formula (3).

### Yield: 49.5%; Purity:99.9%.

### Example 5: Preparation of Indocyanine Green (1)

100 g of formula (2) was dissolved in 100 mL of methanol and stirred for 5-10 min. To this reaction mixture, 63.7 g of formula (4) and 5.2 mL of triethylamine were added and heated to 60-65° C. for 1.5 hrs. The reaction mixture was cooled to 10-15° C. and a solution of 27.6 g of sodium iodide dissolved in 500 mL of methanol, was added. The reaction mixture was heated at 60-65° C. for 1.5 hrs, then cooled to 25-30° C. The reaction mixture was distilled off under vacuum and the solid was filtered and taken in 2000 mL of acetone. The resulting mixture was heated for 1 hr. at 50-60° C. and filtered the solid under hot condition. The solid was washed with 200 mL of acetone and dried under vacuum to obtain crude Indocyanine green of formula (1). Yield: 100 g.

### Example 6: Purification of Indocyanine Green (1)

100 g of crude Indocyanine green of formula (1) was taken in a mixture of 400 mL of methanol and 600 mL of isopropyl alcohol. The reaction mixture was heated for 1 hr at 60-80° C. and filtered the solid under hot condition at 60 to 75° C. The filtered solid was washed with 200 mL of isopropyl alcohol and dried under vacuum to obtain pure Indocyanine green of formula (1). Yield: 42%; Purity by HPLC: 99.84%.

### Example 7: Alternative Process for the Purification of Indocyanine Green (1)

100 g of Indocyanine green of formula (1) was taken in 204 mL of acetone, stirred for 5-10 min and heated to 55-60° C. for 1 hr. The hot reaction mixture was filtered, cooled and the solid formed was washed with 200 mL of acetone. The solid so obtained was further treated with a mixture of acetone and methanol and stirred for 5-10 min. The reaction mixture was heated to 55-65° C. and filtered. The filtrate was cooled and the solid so formed was washed with 200 mL of isopropyl alcohol and dried to obtain pure Indocyanine green of formula (1). Yield: 50%; Purity by HPLC: 99.77%.

Chromatographic conditions:

A high-performance liquid chromatography equipped with Ultraviolet Spectrophotometer as detector and an auto sampler.

| Column | Inertsil ODS 3 V (4.6 × 250 mm, 5μ) | |
|---|---|---|
| Wavelength | 205 | nm |
| Flow rate | 1.0 | ml/minute |
| Injection Volume | 10.0 | μL |
| Column temperature | 25° C. | |
| Run time | 45 | minutes |

| | |
|---|---|
| Diluent | methanol |

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

We claim:

1. A process for the preparation of a substantially pure crystalline Indocyanine green compound of formula (1) 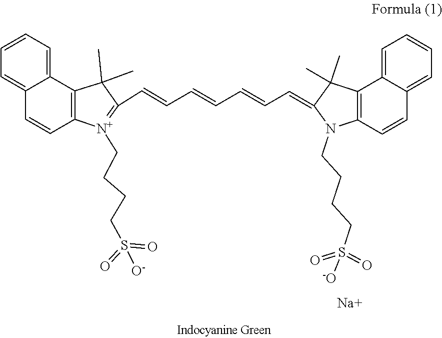
wherein the substantially pure crystalline Indocyanine green compound has a purity greater than 99.0% by HPLC, the process comprising:
a) reacting 1,1,2-trimethyl-1H-benzo[e]indole of formula (5) 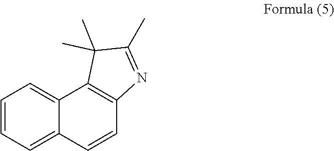 [SMILES: CC1=Nc2ccc3ccccc3c2C1(C)C]
with 1,4-butane sultone to form 4-(1,1,2-trimethyl-1H-benzo[e]indolium-3-yl) butane-1-sulfonate of formula (4); 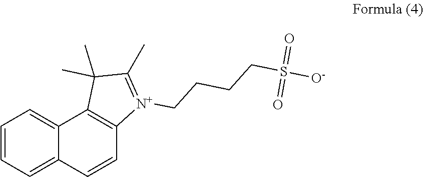 [SMILES: CC1=[N+](CCCCS(=O)(=O)[O-])c2ccc3ccccc3c2C1(C)C]
b) treating formula (4) with E-N-((2E,4E)-5-(phenyl amino) penta-2,4-dienylidene) aniline hydrochloride of formula (3) 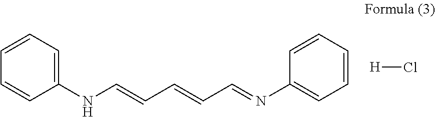
in presence of acetic anhydride to form 4-(1,1-dimethyl-2-((1E,3E,5E)-6-(N-phenyl acetamido) hexa-1,3,5-trienyl)-1H-benzo[e]indolium-3-yl) butane-1-sulfonate of formula (2); 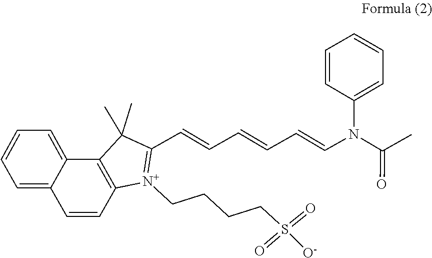 [SMILES: CC(=O)N(/C=C/C=C/C=C/C1=[N+](CCCCS(=O)(=O)[O-])c2ccc3ccccc3c2C1(C)C)c1ccccc1]
c) reacting formula (2) with formula (4) in presence of a base to obtain 4-amino-N-(2-(diethylamino)ethyl)benzamide hydrochloride of formula (1); and
d) purifying 4-amino-N-(2-(diethylamino)ethyl)benzamide hydrochloride or Indocyanine green of formula (1), wherein the purification process comprises:
1) suspending Indocyanine green of formula (1) in a suitable solvents or mixtures thereof;
2) heating the reaction mixture at suitable temperature;
3) filtering the reaction mixture at suitable temperature; and
4) isolating from a suitable solvents or mixture thereof.

2. The process according to claim 1, wherein the base used in step c) is selected from a group consisting of triethylamine, pyridine, ammonia, potassium carbonate, sodium carbonate, potassium bicarbonate, sodium bicarbonate, potassium hydroxide or sodium hydroxide.

3. The process according to claim 1, wherein the solvent used in step d) is selected from a group consisting of acetone, methanol, ethanol, isopropyl alcohol (IPA), n-propanol, n-butanol, or mixtures thereof.

4. A process for the purification of a substantially pure crystalline Indocyanine green compound of formula (1), 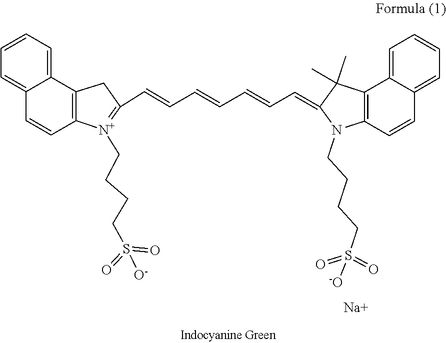
the process comprising:
1) suspending Indocyanine green of formula (1) in a suitable solvents or mixture thereof;
2) heating the reaction mixture at suitable temperature;
3) filtering the reaction mixture at suitable temperature; and
4) isolating the substantially pure crystalline Indocyanine green compound from the suitable solvents or mixture thereof.

5. The process according to claim 4, wherein the solvent is selected from the group consisting of acetone, methanol, ethanol, isopropyl alcohol (IPA), n-propanol, n-butanol, and mixtures thereof.

6. The process according to claim 4, wherein the suitable solvents or mixture there is
a mixture of methanol and isopropyl alcohol;
the suitable temperature for heating is 60-80° C.; and
the suitable temperature for filtering is 60-75° C.

7. The process according to claim 1, wherein preparation of formula (3) comprises the following steps:
i. reacting 1-chloro-2,4-dinitrobenzene of formula (7) 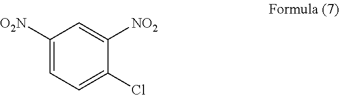 [SMILES: O=[N+]([O-])c1ccc(Cl)c([N+](=O)[O-])c1]
with pyridine to form 1-(2,4-dinitrophenyl)pyridinium chloride of formula (6); 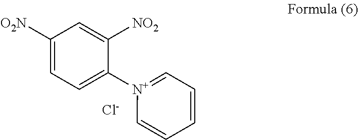 [SMILES: O=[N+]([O-])c1ccc(-[n+]2ccccc2)c([N+](=O)[O-])c1 ; [Cl-]]
ii. reacting formula (6) with methanolic aniline to form N-((2E,4E)-5-(phenyl amino) penta-2,4-dienylidene) aniline hydrochloride of formula (3); and 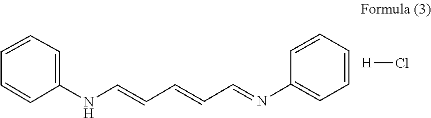
iii. purifying N-((2E,4E)-5-(phenyl amino) penta-2,4-dienylidene) aniline hydrochloride of formula (3).
